(19) United States
(12) Patent Application Publication
(10) Pub. No.: US 2023/0099089 A1
(43) Pub. Date: Mar. 30, 2023

(54) ANTIVIRAL SUBSTANCES WITH A WIDE SPECTRUM OF ACTIVITY

(71) Applicant: Universitaet zu Luebeck Koerperschaft des Oeffentlichen Rechts, Luebeck (DE)

(72) Inventors: Rolf Hilgenfeld, Luebeck (DE); Daizong Lin, Changchun, Jilin (CN); Linlin Zhang, Luebeck (DE)

(21) Appl. No.: 17/798,346

(22) PCT Filed: Feb. 9, 2021
(86) PCT No.: PCT/DE2021/100129

§ 371 Date: Aug. 9, 2022

(30) Foreign Application Priority Data
Feb. 11, 2020 (DE) 10 2020 103 516.0

(51) Int. Cl. C07D 401/12; A61P 31/14

(52) U.S. Cl. CPC C07D 401/12; A61P 31/14

(57) ABSTRACT

A substance of the formula (1) and its use as an antiviral active substance.

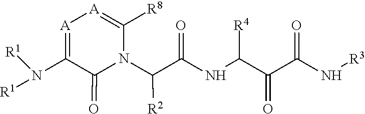

The invention relates to substances with a wide spectrum of activity against 3C - or 3C-like (3CL) proteases of RNA viruses, in particular coronaviruses, picornaviruses (especially enteroviruses) and noroviruses.

RNA viruses, particularly coronaviruses, picornaviruses, and noroviruses, have led to epidemics and pandemics several times in the past decades; for example, the SARS coronavirus broke out in southern China in 2003 and spread to nearly 30 countries around the world. Nine years later, the MERS coronavirus emerged, and in early 2020, the novel coronavirus SARS-CoV-2 formerly also called 2019-nCoV.

The 3C- or 3C-like (3CL) proteases of the RNA viruses in question are seen as promising targets in the development of antiviral agents with broad-spectrum activity.

In recent years, X-ray crystallographic studies have succeeded in characterizing the three-dimensional structure of target systems, providing valuable clues to the structure and nature of potential antiviral agents. [EP16233028B1], [R. Hilgenfeld. “From SARS to MERS: crystallographic studies on coronaviral proteases enable antiviral drug design”, FEBS Journal 281 (2014) p.4085].

The efficacy of α-ketoamides as antiviral agents targeting 3C- or 3C-like (3CL) proteases includes Kim et al. “Broad-Spectrum Antivirals against 3C or 3C-Like Proteases of Picornaviruses, Noroviruses, and Coronaviruses” Journal of Virology, Volume 86 Number 21, p. 11754-11762 (2012).

Crystallography studies show that the α-ketoamide group in the pocket of the target protease (3C or 3CL) interacts at position P1, while between P2 and P3 an amide group is capable of interacting with host cell proteases. It may therefore be a promising strategy to increase the stability of antiviral agents by protecting this very amide group from premature degradation.

US 2015/0133368 A1 and Groutas et al. “Structure-guided design, synthesis and evaluation of oxazolidinone-based inhibitors of norovirus 3CL protease” European Journal of Medicinal Chemistry, Volume 143, 1 Jan. 2018, Pages 881-890 solve the problem via an aliphatic ring closure e.g. via an oxazolidinone, an aliphatic 5-membered ring that carries another oxygen atom in addition to the nitrogen and the carbonyl group adjacent to it, or via the aliphatic 6-membered rings 2,6-piperidinedione or 2,6-diketo-1,3-diazane. The results known so far show a need for optimization, especially with regard to the stability and bioavailability of these compounds.

In WO97/31939 and DE 698 23 178 T2 of the inventor A. E. Adang, inhibitors of serine proteases, in particular thrombin inhibitors, are described, which are built up from sulfone groups and various amino acid residues and, in the case of WO97/31939, have piperidine as side chain.

In Wilmouth et al, Tetrahedron 65 (2009) 2689, the synthesis of a thrombin inhibitor corresponding to DE 698 23 178 T2 (Compound 1) and in Adang et al, Bioorg. Med. Chem. Lett. 9 (1999) 1227, of an inhibitor (compound 35a) corresponding to W097/31939 is described, both thrombin inhibitors having a 2-pyridone in the main chain.

Furthermore, having agents with pulmonary tropism is also an advantage, especially in light of the current coronavirus outbreak.

Therefore, it is the task of the invention to provide new antiviral agents.

More particularly, it is an object of the invention to provide novel antiviral active substances that act on 3C- or 3C-like (3CL) proteases of RNA viruses.

In particular, it is an object of the invention to provide new antiviral active substances with improved bioavailability.

Furthermore, it is an object of the invention to provide new antiviral active substances with pulmonary tropism.

The task of the invention is solved by substances of the following formula or their pharmaceutically acceptable salts and/or adducts and/or tautomers and/or solvates.

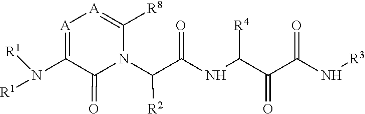

Where A may be the same or different and is selected from the group consisting of N and CR⁸ and where R⁸ is selected from the group consisting of H, F, Cl and Br and where R¹ may be the same or different and is selected from the group consisting of H, alkyl, C(O)OR⁵ wherein R⁵ can be branched or unbranched alkyl, cycloalkyl, substituted or unsubstituted aryl, heteroaryl, arylalkyl, aryloxy, heteroalkyloxy, arylalkoxy, heteroalkylalkoxy; and C(O)NHR⁶ wherein R⁶ can be branched or unbranched alkyl, cycloalkyl, substituted or unsubstituted aryl, heteroaryl, arylalkyl, aryloxy, heteroalkyloxy, arylalkoxy, heteroalkylalkoxy; and SO2R⁷ wherein R⁷ can be branched or unbranched alkyl, cycloalkyl, substituted or unsubstituted aryl, heteroaryl, arylalkyl, aryloxy, heteroalkyloxy, arylalkoxy, heteroalkylalkoxy
and wherein R² is selected from the group consisting of branched or unbranched alkyl, cycloalkyl, cycloalkylmethyl, cycloalkylethyl, cycloalkylpropyl, substituted or unsubstituted aryl, heteroaryl, arylalkyl, aryloxy, heteroalkyloxy, arylalkoxy, heteroalkylalkoxy, and branched or unbranched amino acids
and wherein R³ is selected from the group consisting of H, branched or unbranched alkyl, cycloalkyl, cycloalkylmethyl, cycloalkylethyl, cycloalkylpropyl, aryl, heteroaryl, arylmethyl, heteroarylmethyl,
and wherein R⁴ is selected from the group consisting of

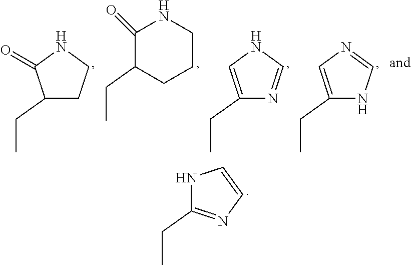

In a particular embodiment, the task of the invention is solved by substances of the following formula or their pharmaceutically acceptable salts and/or adducts and/or tautomers.

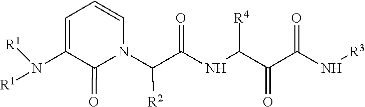

Where R¹ may be the same or different and is selected from the group consisting of H, alkyl, C(O)OR⁵ where R⁵ may be branched or unbranched alkyl, cycloalkyl, substituted or unsubstituted aryl, heteroaryl, arylalkyl, aryloxy, heteroalkyloxy, arylalkoxy, heteroalkylalkoxy; and C(O)NHR⁶ wherein R⁶ can be branched or unbranched alkyl, cycloalkyl, substituted or unsubstituted aryl, heteroaryl, arylalkyl, aryloxy, heteroalkyloxy, arylalkoxy, heteroalkylalkoxy; and SO₂R⁷ wherein R⁷ may be branched or unbranched alkyl, cycloalkyl, substituted or unsubstituted aryl, heteroaryl, arylalkyl, aryloxy, heteroalkyloxy, arylalkoxy, heteroalkylalkoxy

and wherein R² is selected from the group consisting of branched or unbranched alkyl, cycloalkyl, cycloalkylmethyl, cycloalkylethyl, cycloalkylpropyl, substituted or unsubstituted aryl, heteroaryl, arylalkyl, aryloxy, heteroalkyloxy, arylalkoxy, heteroalkylalkoxy, and branched or unbranched amino acids
and wherein R³ is selected from the group consisting of H, branched or unbranched alkyl, cycloalkyl, cycloalkylmethyl, cycloalkylethyl, cycloalkylpropyl, aryl, heteroaryl, arylmethyl, heteroarylmethyl,
and wherein R⁴ is selected from the group consisting of

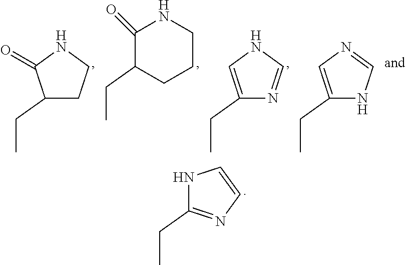

In another particular embodiment, the task of the invention is solved by the following compounds and/or salts, adducts, tautomers, diastereomers and/or solvates of these compounds.

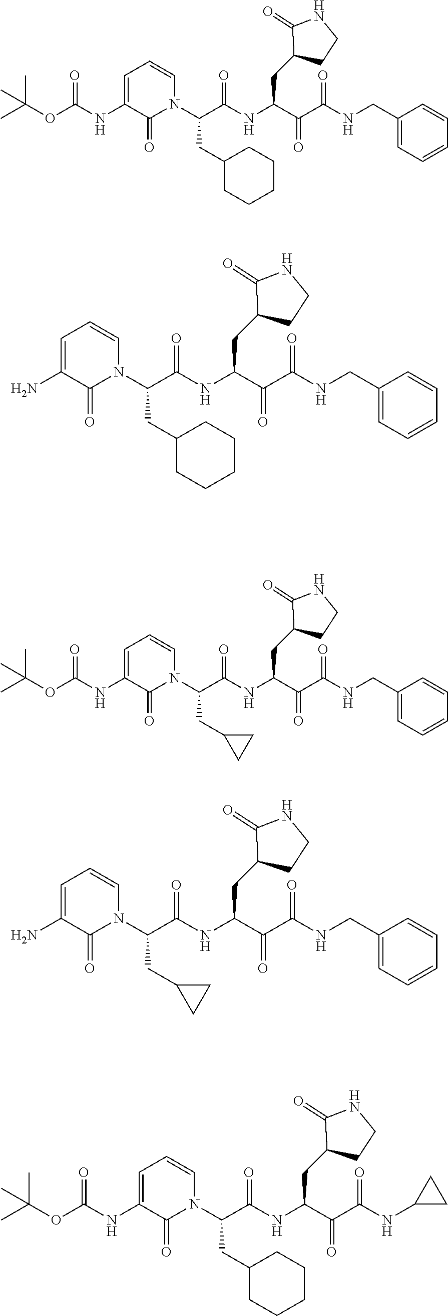 [SMILES: CC(C)(C)OC(=O)Nc1cccn([C@@H](CC2CCCCC2)C(=O)N[C@@H](C[C@@H]2CCNC2=O)C(=O)C(=O)NCc2ccccc2)c1=O ; Nc1cccn([C@@H](CC2CCCCC2)C(=O)N[C@@H](C[C@@H]2CCNC2=O)C(=O)C(=O)NCc2ccccc2)c1=O ; CC(C)(C)OC(=O)Nc1cccn([C@@H](CC2CC2)C(=O)N[C@@H](C[C@@H]2CCNC2=O)C(=O)C(=O)NCc2ccccc2)c1=O ; Nc1cccn([C@@H](CC2CC2)C(=O)N[C@@H](C[C@@H]2CCNC2=O)C(=O)C(=O)NCc2ccccc2)c1=O ; CC(C)(C)OC(=O)Nc1cccn([C@@H](CC2CCCCC2)C(=O)N[C@@H](C[C@@H]2CCNC2=O)C(=O)C(=O)NC2CC2)c1=O]

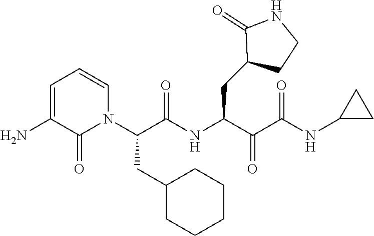 [SMILES: Nc1cccn([C@@H](CC2CCCCC2)C(=O)N[C@@H](C[C@@H]2CCNC2=O)C(=O)C(=O)NC2CC2)c1=O]

In another particular embodiment, the task of the invention is solved by a substance that acts on 3C- or 3C-like (3CL) proteases of RNA viruses.

In another particular embodiment, the task of the invention is solved by a substance acting on the 3CL protease of the coronavirus SARS-CoV-2.

In a further preferred embodiment, the task of the invention is solved by a substance which has a half-life in plasma of more than 30 minutes, preferably more than 40 minutes, particularly preferably more than 44 minutes.

In another preferred embodiment, the task of the invention is solved by a substance exhibiting lung entropism.

The lead compound for antiviral a-ketoamides targeting the 3C or 3C-like (3CL) proteases, designated DZL08, has the following structure:

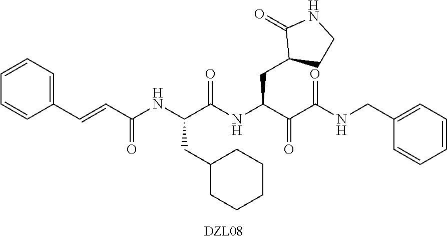 [SMILES: O=C(/C=C/c1ccccc1)N[C@@H](CC1CCCCC1)C(=O)N[C@@H](C[C@@H]1CCNC1=O)C(=O)C(=O)NCc1ccccc1]

The lead compound DZLO8 was tested in virus-infected cell culture and showed good results against enterovirus EV-A71, coxsackievirus B3, and SARS coronavirus and MERS coronavirus (tested in Huh-7 liver cells).

TABLE 1
EC₅₀—values (μM) for the lead substance DZL08

| Host cell type | RD | Huh-T7 | VeroE6 | Vero | Huh7 | Huh7 |
|---|---|---|---|---|---|---|
| Virus | EV-A71 | CVB3 | SARS-CoV | MERS-CoV | MERS-CoV | HCoV-229E |
| DZL08 | 3.7 | 2.8 | 2.1 | 5.0 | 0.0004 | 1.8 |

Crystallography studies show that the a-ketoamide group interacts in the pocket of the target protease at position P1, while the amide group between P2 and P3 is essential to stabilize the complex.

In the organism, the amide group between P2 and P3, in particular, can also be processed by proteases of the host cell, thus reducing the bioavailability of the desired compound.

The invention has now recognized that this amide bond (circle) in DZLO8 can be protected by an aromatic ring closure.

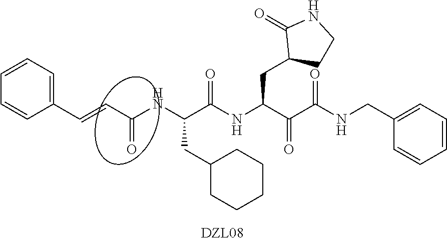 [SMILES: O=C(/C=C/c1ccccc1)N[C@@H](CC1CCCCC1)C(=O)N[C@@H](C[C@@H]1CCNC1=O)C(=O)C(=O)NCc1ccccc1]

The principle is shown using the following substances.

TABLE 2
Principle of protection by aromatic ring formation
based on 6 synthesized active ingredients according to the invention

| | |
|---|---|
| RHCDS1a | 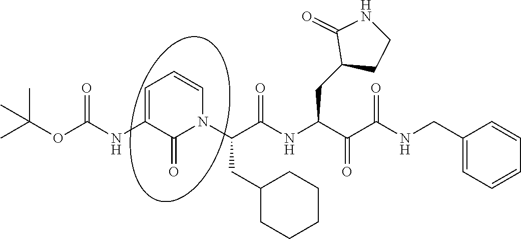 [SMILES: CC(C)(C)OC(=O)Nc1cccn([C@@H](CC2CCCCC2)C(=O)N[C@@H](C[C@@H]2CCNC2=O)C(=O)C(=O)NCc2ccccc2)c1=O] |
| RHCDS1b | 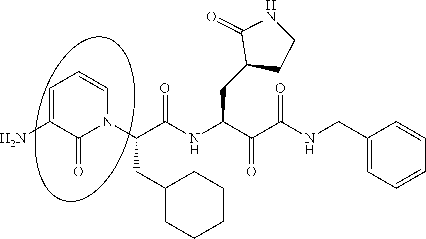 [SMILES: Nc1cccn([C@@H](CC2CCCCC2)C(=O)N[C@@H](C[C@@H]2CCNC2=O)C(=O)C(=O)NCc2ccccc2)c1=O] |
| RHCDS1c | 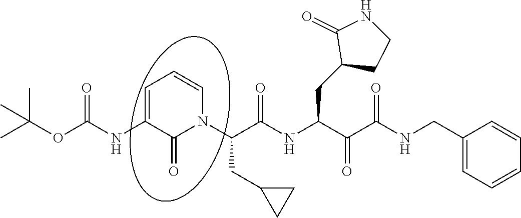 [SMILES: CC(C)(C)OC(=O)Nc1cccn([C@@H](CC2CC2)C(=O)N[C@@H](C[C@@H]2CCNC2=O)C(=O)C(=O)NCc2ccccc2)c1=O] |
| RHCDS1d | 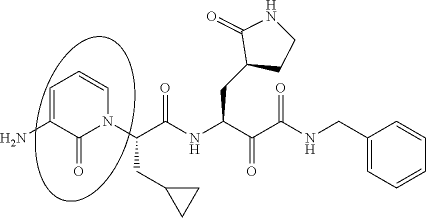 [SMILES: Nc1cccn([C@@H](CC2CC2)C(=O)N[C@@H](C[C@@H]2CCNC2=O)C(=O)C(=O)NCc2ccccc2)c1=O] |
| RHCDS1e | 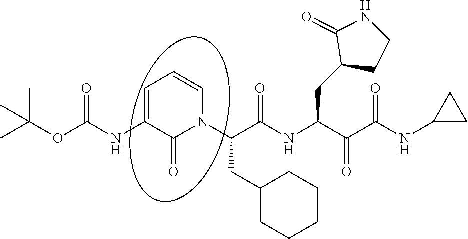 [SMILES: CC(C)(C)OC(=O)Nc1cccn([C@@H](CC2CCCCC2)C(=O)N[C@@H](C[C@@H]2CCNC2=O)C(=O)C(=O)NC2CC2)c1=O] |
| RHCDS1f | 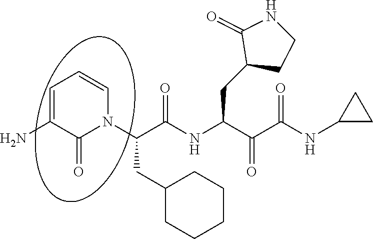 [SMILES: Nc1cccn([C@@H](CC2CCCCC2)C(=O)N[C@@H](C[C@@H]2CCNC2=O)C(=O)C(=O)NC2CC2)c1=O] |

### LIST OF FIGURES

FIG. 1: Complexation of SARS-CoV MP″ by RHCDS1e.

FIG. 2: Complexation of CVB3 3CP″ by RHCDS1e.

FIG. 3: Complexation of MERS-CoV MP″ by RHCDS1c.

FIG. 4: Complexation of the 3CL protease of coronavirus SARS-CoV-2 MP″ by RHCDS1c.

FIG. 5: Pharmacokinetic studies in mice with RHCDS1e.

FIG. 6: Pharmacokinetic studies in mice with RHCDS1e.

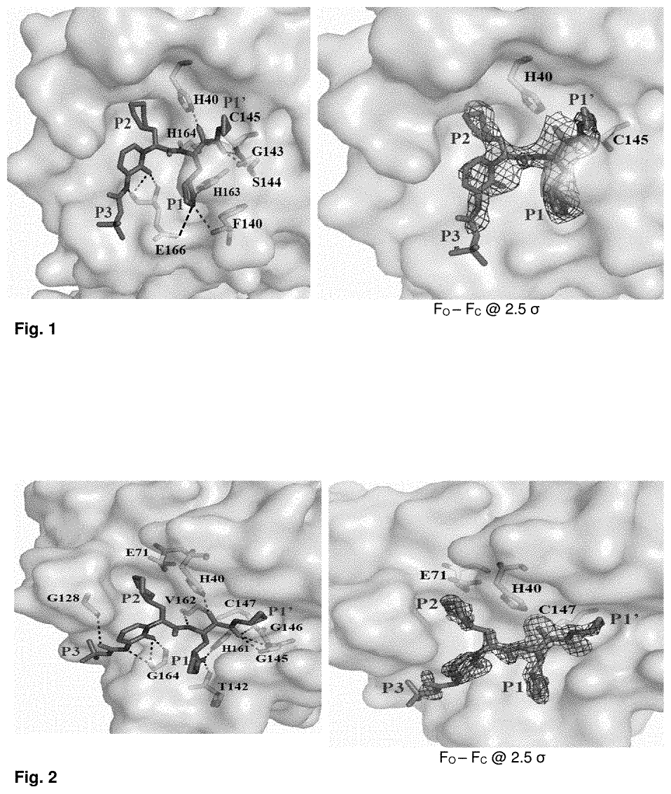

FIG. 1 shows the complexation of SARS coronavirus protease (SARS-CoV M^{pro}) by the drug

RHCD1e based on crystallographic studies.

FIG. 2 shows the complexation of coxsackievirus B3CVB3 3C protease^{pro} by RHCDS1e.

FIG. 3 shows the complexation of MERS coronavirus protease MERS-CoV MP^{pro} by RHCDS1c.

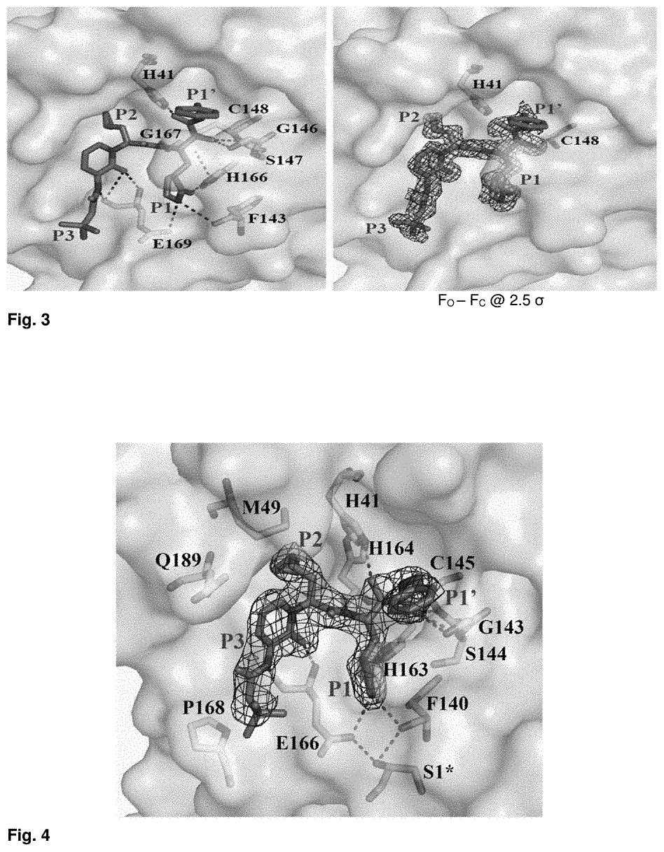

FIG. 4 shows the complexation of 3CL protease of coronavirus SARS-CoV-2 MP″ by RHCDS1c.

The activity of the compounds of the invention compared to the lead compound DZLO8 is particularly good for betacoronavirus proteases (SARS-CoV Mpro, MERS-CoV Mpro, SARS-CoV-2 Mpro). For example, the compound RHCDS1c showed IC50 values of 0.90 uM, 0.58 uM, and 0.67 uM against these proteases (measured by inhibition of cleavage of a standard substrate by the compound in a fluorescence-based assay). These values are slightly improved compared to those obtained with the lead compound DZL08.

The bioavailability of the lead compound DZLO8 was also characterized in detail in an external CRO- study and the results verified.

Pharmacokinetic studies show that the compound RHCDS1e according to the invention has a 50% improved half-life of (t_{1/2}=0.45±0.1 h) compared to the lead compound DZLO8 (t_{1/2}=0.33 ±0.0 h). Stabilization of the P3-P2 amide bond according to the invention also reduced the high plasma protein binding compared to DZLO8 (99%) to 94%.

Table 3 below shows pharmacokinetic data of compound RHCDS1e compared to lead compound DZL08.

TABLE 3
Pharmacokinetic data of RHCDS1e compared to DZL08.

| | DZL08 2 mg/kg i.v. | RHCDS1e 2 mg/kg i.v. |
|---|---|---|
| t1/2 [h] | 0.33 ± 0.0 | 0.45 ± 0.1 |
| V [l/kg] | 8.20 ± 4.1 | 6.73 ± 0.4 |
| CL [ml/min/kg] | 292.43 ± 162.0 | 177.66 ± 51.2 |
| AUC 0-t [ng/ml * h] | 134.65 ± 74.6 | 193.22 ± 59.0 |
| MRI [h] | 0.48 ± 0.0 | 0.47 ± 0.1 |
| CO [ng/ml] | 278.34 ± 138.5 | 461.44 ± 345.0 |

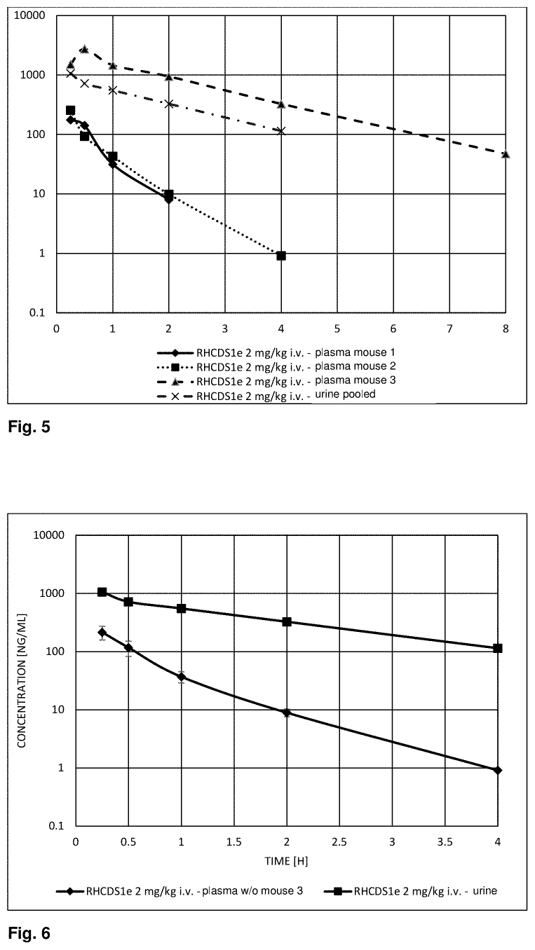

Furthermore, RHCDS1e shows good metabolic stability in mouse and human microsomes. After 30 minutes, 80% of the compound remained metabolically stable in mouse microsomes and 60% in the case of human microsomes. No evidence of toxicity in mice was observed. Pharmacokinetic studies after subcutaneous injection of RHCDS1e in CD-1 mice (20 mg/kg) showed that the compound remained in plasma for only about 4 hours but was excreted in urine for up to 24 hours. The Cmax was 334.50 ng/ml and the mean residence time was approximately 1.59 hours. This can also be seen in FIG. 5 and FIG. 6. Although RHCDS1e disappears from plasma very rapidly, the compound was found at a concentration of 135 ng per g tissue in the lung and 52.7 ng/ml broncheoalveolar washing fluid (BALF) after 24 hours, suggesting a major distribution in the tissue. Given the current coronavirus outbreak, it is advantageous to have agents with pronounced lung tropism.

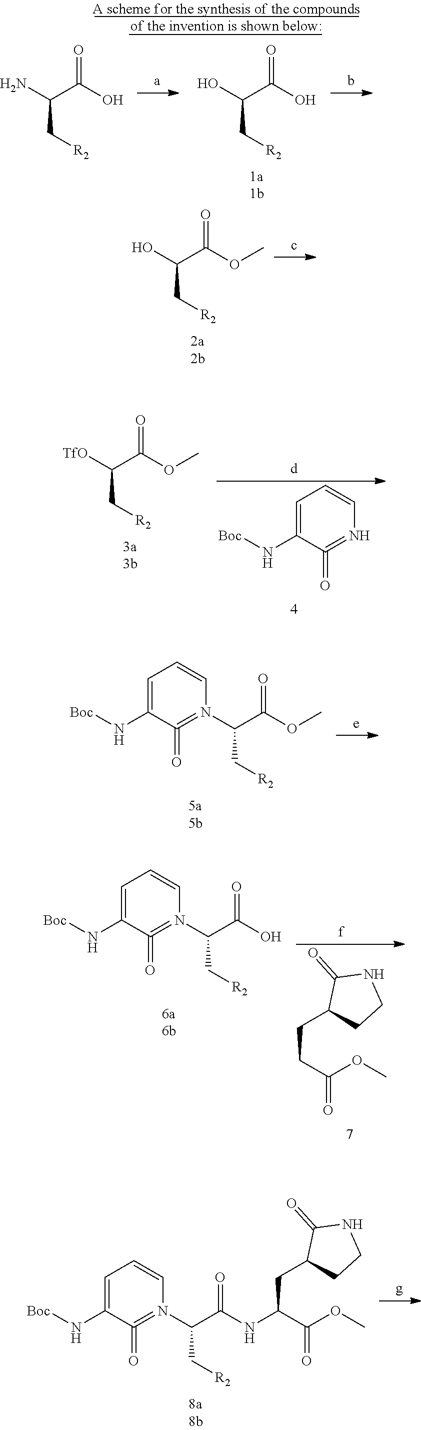

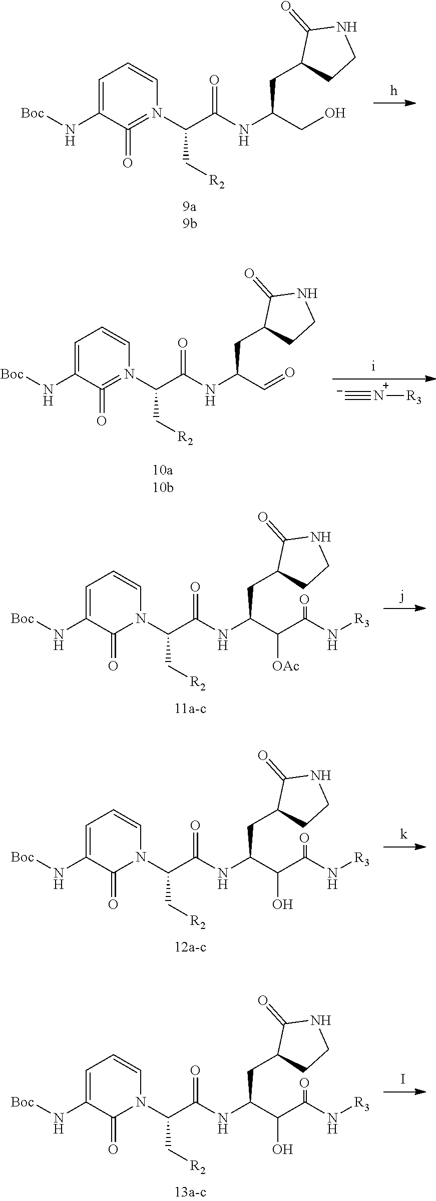

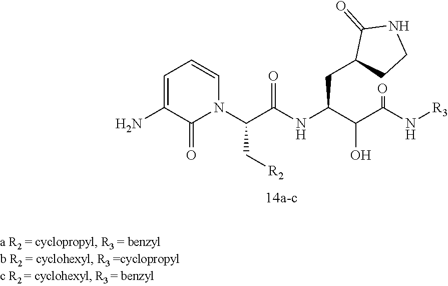

Reaction conditions: (a) NaNO₂, H₂SO₄, H₂O; (b) SOCl₂, MeOH; (c) Tf₂O, 2,6-lutidine, CH₂Cl₂; (d) NaH, THF; (e) LiOH, MeOH, H₂O; (f) HOBT, EDCI, CH₂Cl₂; (g) NaBH₄, MeOH; (h) DMP, NaHCO₃, CH₂Cl₂; (i) isocyanide, AcOH, CH₂Cl₂; (j) LiOH, MeOH, H₂O; (k) DMP, NaHCO₃, CH₂Cl₂; (1) 4 M HCl, EA

In a further aspect of the invention, the agent according to the invention can be used in medicine for therapy of a disease. In particular, for therapy of a disease caused by RNA viruses. Very particularly for therapy of a disease caused by coronaviruses, picornaviruses, including enteroviruses and/or noroviruses.

The active ingredient according to the invention can be supplied in the form of a pharmaceutical preparation.

Such preparations are generally known to the skilled person. In particular, such pharmaceutical preparations and dosage forms are described in US 10,189,810 B2.

### Material and Methods

In the following, not limiting the generality of the teachings, the exemplary preparation of the compounds according to the invention will be described.

### General Procedure

The reagents and reactants used were obtained commercially from commercial sources known to the skilled person and were used without further pretreatment.

HSGF 254 silica gel plates (thickness 0.15-0.2 mm) were used for thin layer chromatography (DC).

All products were characterized by NMR and mass spectroscopy (MS).

¹H-NMR was measured at 300 MHz, chemical shift (δ) is given in ppm, tetramethylsilane was used as standard. The coupling of the protons is characterized as singlet (s), doublet (d), triplet (t), multiplet (m), and broad (br).

A Bruker ESI ion-trap HCT Ultra was used for the MS.

HPLC data were collected using an LC20A (Shimadzu Corporation). Columns: GIST C18 (5 μm, 4.6×150 mm) ternary solvent system (methanol/water, methanol/ 0.1% HCOOH in water, or methanol1/0.1% ammonia in water). Purity was determined by reversed-phase HPLC and was ≥95% for all samples.

### Synthesis of Compound 1

A solution of (R)-2-amino-3-cyclopropylpropanoic acid or (R)-2-amino-3-cyclohexylpropanoic acid (7.74 mmol) in 2N H2504 (15 ml) is stirred at 0° C. Then dropwise NaNO2 (5.34 g, 77.4 mmol) in H₂O (6 ml) is added. The solution is stirred at 0° C. for 3 h and then brought to 20° C. and stirred for 16 h at 20° C. The mixture is extracted with MTBE (50 ml). The organic phase is then dried over anhydrous Na₂SO₄ under vacuum. (Yield of compound 1: 50-75%, colorless oil).

### Synthesis of Compound 2

SOCl₂ (0.8 mL, 11.34 mmol) is added dropwise to a solution of compound 1 (5.72 mmol) in

MeOH (20 ml) at 0° C. The mixture is then stirred for 1.5 h at 20° C. Under vacuum, the solvent is removed and chromatographed over silica gel for purification (PE/EA=1/1). (Yield of compound 2: 30-59%, colorless oil).

### Methyl (R)-3-Cyclopropyl-2-Hydroxypropanoate 2a

¹H NMR (300 MHz, CDC1₃) 6 4.35 (dd, J₁=9.0 Hz, J₂=4.2 Hz, 1 H), 3.80 (s, 3 H), 1.81-1.72 (m, 2 H), 0.92-0.68 (m, 1 H), 0.50-0.43 (m, 2 H), 0.15-0.05 (m, 2 H).

### Methyl (R)-3-Cyclohexyl-2-Hydroxypropanoate 2b

¹H NMR (300 MHz, CDCl ₃) δ4.39-4.34 (m, 1 H), 3.82 (s, 3 H), 1.82-1.48 (m, 8 H), 1.29-1.12 (m, 4 H), 1.00-0.85 (m, 2 H).

### Synthesis of Compound 3

Compound 2 (5.32 mmol) is dissolved in DCM (10 mL) and cooled to 0° C. Portionwise, 2,6-lutidine (1.5 ml, 13.26 mmol) and Tf20 (3.3 g, 11.87 mmol) are added. The mixture is stirred at 0° C. for 30 min. The mixture is washed with saline and 1N HCl (3:1 v/v) and then extracted with MTBE and dried with anhydrous Na₂SO₄ under vacuum. (Yield of compound 3: 82%, brown oil).

### Synthesis of Compound 5

Tert-butyl (2-oxo-1,2-dihydropyridin-3-yl)carbamate (379 mg, 1.8 mmol) is dissolved in THF (15 ml). The NaH (115 mg, 2.80 mmol, 60% in oil) is added at 0° C. and then stirred for 30 min. Compound 3 (515 mg, 1.86 mmol) in THF (10 ml) is added. The mixture is stirred for 20 hat 25° C. Under vacuum, the solvent is removed and chromatographed over silica gel for purification (PE/EA). (Yield of compound 5: 56-60%, light yellow solid).

### Methyl (S)-2-(3-((Tert-Butoxycarbonyl)Amino)-2-Oxopyridin-1(2 H)-yl)-3-Cyclopropylpropanoate 5a

¹H NMR (300 MHz, DMSO-d₆) δ7.83-7.78 (m, 2 H), 7.35 (dd, J₁=7.2 Hz, J₂=1.5 Hz, 1 H), 6.30 (t, J=7.2 Hz, 1 H), 5.36 (dd, J₁=10.8 Hz, J₂=4.5 Hz, 1 H), 3.57 (s, 3 H), 1.81-1.62 (m, 2 H), 1.48 (s, 9 H), 0.55-0.48 (m, 1 H), 0.34-0.29 (m, 2 H), 0.15-0.12 (m, 1 H), 0.04-0.01 (m, 1 H). ESI-MS (m/z): 337 [M +H]⁺.

### Methyl (5)-2-(3-((Tert-Butoxycarbonyl)amino)-2-Oxopyridin-1(2 H)-yl)-3-Cyclohexylpropanoate 5b

¹H NMR (300 MHz, DMSO-d6) δ7.82-7.76 (m, 2 H), 7.35 (dd, J₁=7.5 Hz, J₂=1.5 Hz, 1 H), 6.30 (t, J =7.5 Hz, 1 H), 5.35 (dd, J₁=11.1 Hz, J₂=4.5 Hz, 1 H), 3.56 (s, 3 H), 2.10-1.88 (m, 2 H), 1.78-1.72 (m, 1 H), 1.65-1.44 (m, 13 H), 1.14-0.82 (m, 6 H). ESI-MS (m/z): 379 [M +H]⁺.

### Synthesis of Compound 6

To compound 5 (1.65 mmol) in MeOH (15 ml) and H₂O (3 ml) is added LiOH.H20 (139 mg, 3.31 mmol). The mixture is stirred for 1 h at 20° C. A pH=6˜7 is adjusted with 1 N HC1. Under vacuum, the solvent is removed and chromatographed over silica gel for purification (DCM/MeOH =10/1) (yield of compound 6: 452 mg, 84%, pale yellow solid).

### (S)-2-(3-((Tert-Butoxycarbonyl)Amino)-2-Oxopyridin-1(2 H)-Yl)-3-Cyclopropylpropanoic Acid 6a

¹H NMR (300 MHz, DMSO-d6) 6 13.11(s, 1 H), 7.81-7.77 (m, 2 H), 7.36 (dd, J₁=6.9 Hz, J₂=1.5 Hz, 1 H), 6.30 (t, J=6.9 Hz, 1 H), 5.35 (dd, J₁=10.5 Hz, J₂=4.5 Hz, 1 H), 1.80-1.69 (m, 2 H), 1.47 (s, 9 H), 0.53-0.48 (m, 1 H), 0.32-0.29 (m, 2 H), 0.14-0.11 (m, 1 H), 0.03-0.00 (m, 1 H). ESI-MS (m/z): 323 [M +H]⁺.

### (S)-2-(3-((Tert-Butoxycarbonyl)Amino)-2-Oxopyridin-1(2 H)-yl)-3-Cyclohexylpropanoic Acid 6b

¹H NMR (300 MHz, DMSO-d6) 6 13.12 (s, 1 H), 7.83-7.77 (m, 2 H), 7.35 (dd, J₁=7.2 Hz, J_{2 =1.5} Hz, 1 H), 6.30 (t, J =7.2 Hz, 1 H), 5.35 (dd, J₁=10.8 Hz, J₂ =4.5 Hz, 1 H), 2.10-1.92 (m, 2 H), 1.78-1.69 (m, 1 H), 1.65-1.52 (m, 4 H), 1.47 (s, 9H), 1.13-0.82 (m, 6 H). ESI-MS (m/z): 365 [M +H]⁺.

### Synthesis of Compound 8

HOBT (245 mg, 1.82 mmol) and EDCI (349 mg, 1.82 mmol) are added to a solution of compound 6 (1.65 mmol) in DCM (20 ml). The mixture is stirred for 1 hat 0° C. Compound 7 methyl (S)-2-amino-3-((S)-2-oxopyrrolidin-3-yl)propanoate (307 mg, 1.65 mmol) is then added, and pH =9 is adjusted with Et3N. The mixture is stirred for 24 h at 0° C. Under vacuum, the solvent is removed and chromatographed over silica gel for purification (DCM/MeOH =20/1) (yield of compound 8: 59-67%, light yellow solid).

### Methyl (S)-2-((S)-2-(3-((Tert-Butoxycarbonyl)Amino)-2-Oxopyridin-1(2 H)-Yl)-3-Cyclopropylpropanamido)-3-((S)-2-Oxopyrrolidin-3-yl)Propanoate 8a

¹ H NMR (300 MHz, DMSO-d6) 6 9.00-8.92 (m, 1 H), 7.81-7.77 (m, 2 H), 7.37-7.34 (m, 1 H), 6.30 (t, J=7.2 Hz, 1 H), 5.77 (dd, J₁=10.8 Hz, J₂=4.5 Hz, 1 H), 4.54-4.45 (m, 1 H), 3.74 (s, 3 H), 3.37-3.29 (m, 2 H), 2.35-2.25 (m, 2 H), 1.90-1.71 (m, 5 H), 1.46 (s, 9H), 0.51-0.46 (m, 1 H), 0.32-0.29 (m, 2 H), 0.15-0.11 (m, 1 H), 0.04-0.00 (m, 1 H). ESI-MS (m/z): 491 [M +H]⁺.

### Methyl (S)-2-((S)-2-(3-((Tert-Butoxycarbonyl)amino)-2-Oxopyridin-1(2 H)-yl) Cyclohexylpropanamido)-3-((S)-2-Oxopyrrolidin-3-yl)Propanoate 8b

ESI-MS (m/z): 533 [M+H]⁺.

### Synthesis of Compound 9

NaBH₄ (200 mg, 5.3 mmol) is added to a solution of compound 8 (0.53 mmol) in MeOH (6 ml). The mixture is stirred for 3 h at 25° C. Under vacuum, the solvent is removed and chromatographed over silica gel for purification (DCM/MeOH=10/1) (yield of compound 9: 49%, approximately white solid).

### Tert-Butyl(1-((S)-3-Cyclopropyl-1-(((S)-1-Hydroxy -3-(( )2-Oxopyrrolidin-3-yl)Propan-2-Yl)Amino)-1-Oxopropan-2-Yl)-2-oxo-1,2-Dihydropyridin-3-Yl)Carbamate 9a

ESI-MS (m/z): 463 [M +H]⁺.

### Tert-butyl(1-((S)-3-Cyclohexyl-1-(((S)-1-Hydroxy-3-((S)-2-Oxopyrrolidin-3-Yl)Propan-2-Yl)Amino)-1-Oxopropan-2-yl)-2-Oxo-1,2-Dihydropyridin-3-Yl)Carbamate 9b

ESI-MS (m/z): 505 [M +H]⁺.

### Synthesis of Compound 10

Dess-Martin periodinan (116 mg, 0.27 mmol) and NaHCO₃ (8 mg, 0.09 mmol) are added to a solution of compound 9 (0.26 mmol) in DCM (15 ml). The mixture is stirred for 1 h at 20° C. Under vacuum, the solvent is removed and chromatographed over silica gel for purification (DCM/MeOH =20/1) (yield of compound 10: 83-90%, approximately white solid).

### Tert-butyl(1-((S)-3-Cyclopropyl-1-Oxo-1-(((S)-1-Oxo -34(S)-2-Oxopyrrolidin-3-Yl)Propan-2-Yl)Amino)Propan-2-Yl)-2-Oxo-1,2-Dihydropyridin-3-Yl)carbamate 10a

¹ H NMR (300 MHz, DMSO-d6) 6 9.40 (d, J =7.8 Hz, 1 H), 8.97 (dd, JI =14.1 Hz, J₂=7.2 Hz, 1 H), 7.79-7.73 (m, 2 H), 7.35-7.32 (m, 1 H), 6.30 (t, J =7.5 Hz, 1 H), 5.69-5.62 (m, 1 H), 4.48-4.42 (m, 1 H), 3.20-3.10 (m, 2 H), 2.32-2.15 (m, 2 H), 1.88-1.66 (m, 5 H), 1.46 (s, 9H), 0.55-0.47 (s, 1 H), 0.36-0.29 (m, 2 H), 0.14-0.11 (m, 1 H), 0.04-0.00 (m, 1 H). ESI-MS (m/z): 461 [M +H]⁺. Tert-butyl(1-((S)-3-cyclohexyl-1-oxo-1-(((S)-1-oxo-3-((S)-2-oxopyrrolidin-3-yl)propan-2-yl)amino)propan-2-yl)-2-oxo-1,2- dihydropyridin-3-yl)carbamate 10b:

ESI-MS (m/z): 503 [M +H]⁺.

### Synthesis of Compound 11 (General Method)

Acetic acid (26 mg, 0.44 mmol) and isocyanide (0.22 mmol) are added to a solution of compound 10 (0.22 mmol) in DCM (15 ml). The mixture is stirred for 24 h at 20° C. Under vacuum, the solvent is removed and chromatographed over silica gel for purification (DCM/MeOH =20/1) (yield of compound 11: 57-65%, approximately white solid).

### Synthesis of Compound 12 (General Method)

To compound 11 (0.13 mmol) in MeOH (15 mL) and H₂O (3 mL) is added LiOH.H2O (11 mg, 0.26 mmol). The mixture is stirred for 20 min at 20° C. A pH=6-7 is adjusted with 1 N HCl. Under vacuum, the solvent is removed and chromatographed over silica gel for purification

(DCM/MeOH =10/1) (yield of compound 12: 90-95%, approximately white solid).

### Synthesis of Compound 13 (General Method)

Compound 12 (0.115 mmol) is dissolved in DCM (15 ml), Dess-Martin periodinan (58 mg, 0.14 mmol) and NaHCO₃ (4 mg, 0.05 mmol) are added. The mixture is stirred for 1 h at 25° C.

Under vacuum, the solvent is removed and chromatographed over silica gel for purification (DCM/MeOH =10/1) (yield of compound 13: 69-80%, approximately white solid).

### Tert-butyl(1-((S)- 1-(((S)-4-(Benzylamino)- 3 ,4-Dioxo-1-((S)-2-oxopyrrolidin-3-yl)butan-2-Yl)Amino)-3-Cyclopropyl-1-Oxopropan-2-Yl)-2-Oxo-1, 2-dihydropyridin-3-yl)carbamate 13a

¹ H NMR (300 MHz, DMSO-d₆) δ9.25 (d, J=5.4 Hz, 1 H), 9.00 (dd, J₁=14.1 Hz, J₂=7.2 Hz, 1 H), 7.79-7.69 (m, 3 H), 7.35-7.22 (m, 5 H), 6.30-6.24 (m, 1 H), 5.69-5.61 (m, 1 H), 4.97 (s, br, 1 H), 4.29 (s, 2 H), 3.17-3.09 (m, 2 H), 2.30-2.15 (m, 2 H), 1.91-1.62 (m, 5 H), 1.46 (s, 9H), 0.52-0.47 (m, 1 H), 0.33-0.29 (m, 2 H), 0.14-0.11 (m, 1 H), 0.05-0.02 (m, 1 H). ESI-MS (m/z): 594 [M +H]⁺.

### Tert-butyl(1-((S)-3-Cyclohexyl- 1-((( )4-(Cyclopropylamino)-3 ,4-Dioxo -1-((S)-2-Oxopyrrolidin-3-Yl)Butan-2-yl)Amino)-1-Oxopropan-2-Yl)-2-Oxo-1,2-Dihydropyridin-3-Yl)Carbamate 13b

¹ H NMR (300 MHz, CDCl₃) δ8.78-8.50 (m, 1 H), 8.01-7.92 (m, 1 H), 7.65 (d, J=7.5 Hz, 1 H), 7.10-6.90 (m, 2 H), 6.35-6.14 (m, 2 H), 5.85-5.75 (m, 1 H), 5.25-5.10 (m, 1 H), 3.24-3.13 (m, 2 H), 2.75-2.71 (m, 1 H), 2.49-2.20 (m, 1 H), 2.10-1.81 (m, 3 H) 1.80-1.52 (m, 8 H), 1.49 (s, 9H), 1.25-1.01 (m, 4 H), 1.00-0.73 (m, 4 H), 0.56-0.51 (m, 2 H). ESI-MS (m/z): 586 [M +H]⁺.

### Tert-butyl(1-((S)- 1-(((S)-4-(benzylamino)-3 ,4-dioxo -1-((S)-2-Oxopyrrolidin-3-Yl)Butan Yl)amino)-3-Cyclohexyl-l-Oxopropan-2-Yl)-2-Oxo-1,2-Dihydropyridin-3-Yl)Carbamate 13c

¹ H NMR (300 MHz, CDCl₃) 6 8.74-8.25 (m, 1 H), 7.99-7.91 (m, 1 H), 7.65 (d, J =7.5 Hz, 1 H), 7.33-7.00 (m, 6 H), 6.26 (t, J =7.5 Hz, 1 H), 6.07 (s, 1 H), 5.84-5.73 (m, 1 H), 5.27-5.19 (m, 1 H), 4.48-4.42 (m, 2 H), 3.40-3.30 (m, 2 H), 2.52-2.29 (m, 2 H), 2.10-1.92 (m, 3 H) 1.79-1.53 (m, 7H), 1.50 (s, 9H), 1.24-1.01 (m, 4 H), 1.00-0.76 (m, 2 H), ESI-MS (m/z): 636 [M +H]⁺.

### Synthesis of compound 14 (General Method)

To compound 13 (0.11 mmol) is added a solution of 4N HC1/EA (30 ml). The mixture is stirred for 1 h at 25° C.Then, under reduced pressure, the solvent is removed and diethyl ether (2 ml) is added to the residue with stirring. The white solid is precipitated, filtered, and the filter cake is washed with diethyl ether (1 mL). (Yield of compound 14: 58-94%, white solid).

### (S)-3-((S)-2-(3-Amino-2-oxopyridin-1(2 H)-Yl)-3-Cyclopropylpropanamido)-N-benzyl-2-Oxo-4-((S)-2-Oxopyrrolidin-3-Yl)Butanamide 14a

¹ H NMR (300 MHz, DMSO-d6) 6 9.25 (d, J =5.4 Hz, 1 H), 9.06 (dd, J₁=14.1 Hz, J₂=7.2 Hz, 1 H), 7.73-7.59 (m, 2 H), 7.34-7.21 (m, 5 H), 6.30 (t, J =7.5 Hz, 1 H), 5.75-5.64 (m, 1 H), 5.00 (s, br, 1 H), 4.30 (s, 2 H), 3.17-3.09 (m, 2 H), 2.26-2.12 (m, 2 H), 1.99-1.63 (m, 5 H), 1.46 (s, 9H), 0.49-0.46 (m, 1 H), 0.32-0.30 (m, 2 H), 0.15-0.14 (m, 1 H), 0.05-0.00 (m, 1 H). ESI-MS (m/z): 494 [M +H]t

### (S)-3-((S)-2-(3-Amino-2-Oxopyridin-1(2 H)-Yl)-3-Cyclohexylpropanamido)-N-Cyclopropyl-2-Oxo-4-((S)-2-Oxopyrrolidin-3-Yl)Butanamide 14b

¹ H NMR (300 MHz, DMSO-d6) 6 9.18-9.05 (m, 1 H), 8.78-8.71 (m, 1 H), 7.72 (d, J =7.5 Hz, 1 H), 7.59-7.57 (m, 1 H), 7.40-7.38 (m, 1 H), 6.30 (t, J =7.5 Hz, 1 H), 5.78-5.74 (m, 1 H), 4.96 (s, br, 1 H), 3.28-3.18 (m, 2 H), 2.74-2.71 (m, 1 H), 2.36-2.25 (m, 1 H), 2.10-2.00 (m, 1 H), 1.98-1.77 (m, 3 H) 1.73-1.52 (m, 7 H), 1.10-0.73 (m, 6 H), 0.64-0.55 (m, 4 H). ESI-MS (m/z): 486 [M +H]⁺.

### (S)-3-((S)-2-(3-Amino-2-Oxopyridin-1(2 H)-yl)-3-Cyclohexylpropanamido)-N-Benzyl-2-Xxo-4-((S)-2-Oxopyrrolidin-3-yl)Butanamide 14c

¹H NMR (300 MHz, DMSO-d₆) δ9.29-9.06 (m, 2 H), 7.99-7.91 (m, 1 H), 7.73 (d, J=7.5 Hz, 1 H), 7.52-7.50 (m, 1 H), 7.34-7.23 (m, 6 H), 6.28 (t, J=7.5 Hz, 1 H), 5.81-5.71 (m, 1 H), 5.10-4.98 (m, 1 H), 4.31-4.29 (m, 2 H), 3.29-3.19 (m, 2 H), 2.35-2.17 (m, 2 H), 1.89-1.78 (m, 3 H) 1.67-1.58 (m, 7 H), 1.20-0.79 (m, 6H). ESI-MS (m/z): 536 [M+H]⁺.

1. A substance of the following formula: 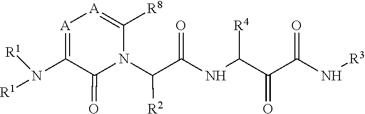
wherein
A may be the same or different and is selected from the group consisting of N and CR⁸ wherein R⁸ is selected from the group consisting of H, F, Cl and Br,
R¹ may be the same or different and is selected from the group consisting of H, alkyl, C(O)OR⁵ wherein R⁵ can be branched or unbranched alkyl, cycloalkyl, substituted or unsubstituted aryl, heteroaryl, arylalkyl, aryloxy, heteroalkyloxy, arylalkoxy, heteroalkylalkoxy; and C(O)NHR⁶ wherein R⁶ can be branched or unbranched alkyl, cycloalkyl, substituted or unsubstituted aryl, heteroaryl, arylalkyl, aryloxy, heteroalkyloxy, arylalkoxy, heteroalkylalkoxy and SO₂R⁷ wherein R⁷ can be branched or unbranched alkyl, cycloalkyl, substituted or unsubstituted aryl, heteroaryl, arylalkyl, aryloxy, heteroalkyloxy, arylalkoxy, heteroalkylalkoxy,
R² is selected from the group consisting of branched or unbranched alkyl, cycloalkyl, cycloalkylmethyl, cycloalkylethyl, cycloalkylpropyl, substituted or unsubstituted aryl, heteroaryl, arylalkyl, aryloxy, heteroalkyloxy, arylalkoxy, heteroalkylalkoxy, and branched or unbranched amino acids,
R³ is selected from the group consisting of H, branched or unbranched alkyl, cycloalkyl, cycloalkylmethyl, cycloalkylethyl, cycloalkylpropyl, aryl, heteroaryl, arylmethyl, heteroarylmethyl, and
R⁴ is selected from the group consisting of 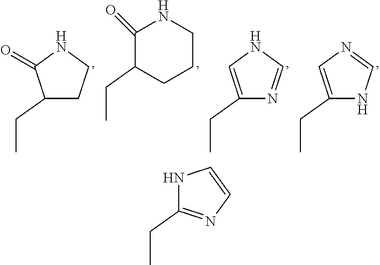
and/or its salts, adducts, tautomers and/or solvates.

2. The substance according to claim 1 of the following formula: 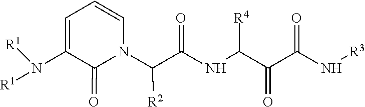
and/or its salts, adducts, tautomers and/or solvates.

3. The substance according to claim 1, wherein it is selected from the group consisting of: 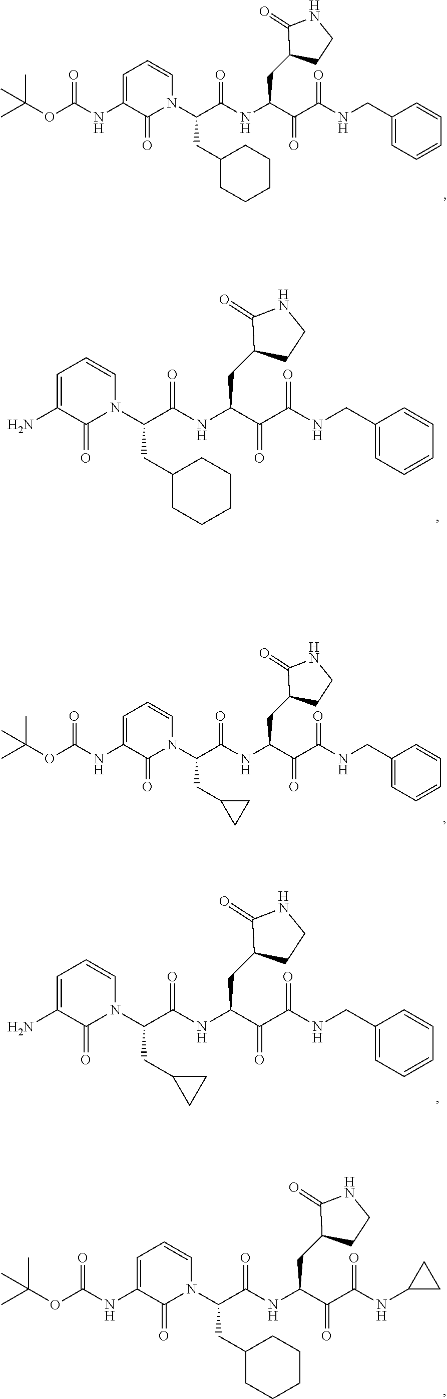 [SMILES: CC(C)(C)OC(=O)Nc1cccn([C@@H](CC2CCCCC2)C(=O)N[C@@H](C[C@@H]2CCNC2=O)C(=O)C(=O)NCc2ccccc2)c1=O ; Nc1cccn([C@@H](CC2CCCCC2)C(=O)N[C@@H](C[C@@H]2CCNC2=O)C(=O)C(=O)NCc2ccccc2)c1=O ; CC(C)(C)OC(=O)Nc1cccn([C@@H](CC2CC2)C(=O)N[C@@H](C[C@@H]2CCNC2=O)C(=O)C(=O)NCc2ccccc2)c1=O ; Nc1cccn([C@@H](CC2CC2)C(=O)N[C@@H](C[C@@H]2CCNC2=O)C(=O)C(=O)NCc2ccccc2)c1=O ; CC(C)(C)OC(=O)Nc1cccn([C@@H](CC2CCCCC2)C(=O)N[C@@H](C[C@@H]2CCNC2=O)C(=O)C(=O)NC2CC2)c1=O] 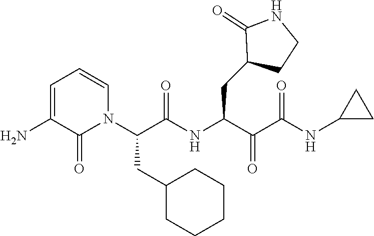 [SMILES: Nc1cccn([C@@H](CC2CCCCC2)C(=O)N[C@@H](C[C@@H]2CCNC2=O)C(=O)C(=O)NC2CC2)c1=O]
and/or salts, adducts, tautomers, diastereomers and/or solvates of these substances.

4. The substance according to claim 1, wherein it acts on 3C- or 3C-like (3CL)-proteases of RNA viruses.

5. The substance according to claim 1, wherein it acts on the 3CL protease of the coronavirus SARS-CoV-2.

6. The substance according to claim 1, wherein it has a half-life in plasma of more than 30 minutes.

7. The substance according to claim 1, wherein it exhibits lung entropism.

8. A medicine comprising the substance according to claim 1 in a pharmaceutically acceptable carrier.

9. A method for treatment of a disease comprising administering to a patient in need thereof a substance according to claim 1.

10. The method according to claim 9, wherein the disease is caused by RNA viruses.

11. The method according to claim 9, characterized in that wherein the disease is caused by at least one of coronaviruses, picornaviruses, enteroviruses and noroviruses.

12. The method according to claim 9, characterized in that wherein the disease is caused by SARS-CoV-2.

13. The substance according to claim 1, wherein it has a half-life in plasma of more than 40 minutes

14. The substance according to claim 1, wherein it has a half-life in plasma of more than 44 minutes.
